(19) United States
(12) Patent Application Publication
(10) Pub. No.: US 2014/0154315 A1
(43) Pub. Date: Jun. 5, 2014

(54) Hydrophobic Preparations

(71) Applicant: Roger New, London (GB)

(72) Inventor: Roger New, London (GB)

(73) Assignee: VAXCINE LTD., St. Helier (GB)

(21) Appl. No.: 14/115,922

(22) PCT Filed: May 4, 2012
(86) PCT No.: PCT/EP2012/058279

§ 371 Date: Feb. 7, 2014

(30) Foreign Application Priority Data
May 6, 2011 (GB) 1107629.6

(51) Int. Cl. A61K 9/107; A61K 39/00

(52) U.S. Cl. CPC A61K 9/1075; A61K 39/0008
USPC 424/463; 514/786; 424/184.1; 424/209.1; 424/272.1

(57) ABSTRACT

The present invention relates to preparations of substances in hydrophobic solvents in which they would not normally be soluble and to processes for obtaining these preparations. In particular, the invention relates to preparations of hydrophilic species in hydrophobic solvents such as oils. The use of these preparations as vaccines and in pharmaceutical compositions is also described.

The present invention relates to preparations of substances in hydrophobic solvents in which they would not normally be soluble and to processes for obtaining these preparations. In particular, the invention relates to preparations of hydrophilic species in hydrophobic solvents such as oils.

The invention in particular applies to hydrophilic macromolecules that would not normally be soluble in oils or other hydrophobic solvents.

For many applications, e.g. in the pharmaceutical sciences, in food technology or the cosmetics industry, work with proteins and similar macromolecules presents problems because their hydrophilicity and high degree of polarity limit the extent to which they can interact with or incorporate into lipid phases. Many natural systems employ lipidic barriers (e.g. skin, cell membranes) to prevent access of hydrophilic molecules to internal compartments; the ability to disperse proteins in lipidic vehicles would open up a new route to introduction of these macromolecules into biological systems, whereby the lipid medium containing the protein can integrate with the hydrophobic constituents of barriers, instead of being excluded by them.

Another area where incorporation of proteins into oils may confer advantage is for the use of enzymes in organic phases. Enzymic syntheses are becoming increasingly important compared to chemical processes because of their much lower energy needs, greater substrate and product specificities, high yields, and the fact that many reactions are catalysed which are impossible by chemical means. Recent findings that enzymes can remain active in organic environments have opened up many additional possibilities. Thus, reactions involving lipophilic substrates and products may be catalysed effectively, and enzyme stability is often much greater than in aqueous environments, allowing them to be used in much more extreme conditions such as at high temperature. A very important aspect is that reactions involving hydrolytic enzymes such as lipases and peptidases can preferentially go in the reverse direction in low water environments, thus enabling the synthesis of a wide range of industrially important compounds. Another application is where a complex chain of reactions is involved in which the multiple catalytic units need to be maintained in close proximity to each other. Such might be the case in light-initiated redox reactions. An additional possibility is the controlled production of nanoparticulates in oil phase, using enzymes to induce mineralisation by action on organometallic substrates. The preparation of a stable dispersion of preformed nanoparticulates in oil phase may also be advantageous for the performance of certain surface-catalysed reactions.

Dispersion of hydrophilic substances in oil phase rather than aqueous media confers other benefits in terms of increasing their stability with respect to temperature-mediated denaturation, hydrolysis, light sensitivity etc. Oils can be chosen which remain fluid over a wider temperature range than aqueous solutions, or that have a higher viscosity, resulting in greater protection against physical damage. In mixed-phase systems, sequestration of proteins in oil can limit mutually harmful interactions—e.g. oxidation—with water-soluble compounds.

A further advantage of the compositions or preparations of this invention is that are that they are essentially anhydrous and therefore stable to hydrolysis. They are also stable to freeze-thawing and have greater stability at high temperatures, probably because water must be present in order for the protein to unfold and become denatured. This means that they may be expected to have a much longer shelf life than aqueous preparations of the hydrophilic species. In addition, as the preparations are anhydrous they are more compatible with capsules used in pharmaceutical practice, where both gelatin and Hydroxypropyl Methylcellulose (HPMC) capsule shells can take up moisture and soften as a result.

Solubilisation of such materials in incompatible phases can be effected by surrounding them in a sheath of amphiphile which is compatible with both the material being solubilised, and the continuous phase. Such a method has been described in patent application WO96/014871, in which lamella-forming amphiphiles such as phospholipids are dispersed in aqueous phase to form small unilamellar vesicles (SUV liposomes) and then mixed with macromolecules prior to removal of the water by lyophilisation, followed by addition of a hydrophobic (oil) phase. It has been proposed that, during the process of addition of oil, the liposome membranes fuse with each other and form a continuous expanse of uni- or multilamellar membrane (an amphiphile sheath) completely surrounding the macromolecule.

This method has severe limitations, however, since the ratio of amphiphile to solute (wt/wt) required to achieve complete solubilisation, as determined by absence of scattering of visible light, is high. In oils such as oleic acid, the amphiphile/solute ratio is usually ≧7, while triglycerides require between 15-20 times as much amphiphile as solute to achieve satisfactory solubilisation, and in the case of mineral oil, effective solubilisation of high concentrations of macromolecules is generally not possible. Since amphiphiles themselves have limited solubility in triglycerides and other oil phases, this places a severe restriction on the total maximum amount of solute which can be accommodated in the oil phase—usually less than 5 mg/ml. Furthermore, when these oil phases are dispersed in aqueous phase as emulsions, the molecules solubilised therein are readily released into the aqueous phase, resulting in loss of between thirty to seventy percent of the macromolecule, under normal circumstances.

It has now been found that macromolecules can be enclosed within an amphiphile sheath in a much more efficient way than disclosed in WO96/014871. This is brought about firstly by dissolving amphiphile in an organic phase such as cyclohexane, then dispersion of the solution of macromolecule, dissolved in an aqueous phase, in the cyclohexane to form a water-in-oil emulsion. In this way, the macromolecule is surrounded by a single layer of amphiphile, rather than multiple layers, as in WO96/014871. Surprisingly however, it has been discovered that, in order to implement this method, use of a single amphiphile such as soya phosphatidyl choline is not sufficient, and that this method will only work when special combinations of amphiphiles in specific proportions are employed. A person skilled in the art, therefore, would not be able to arrive at the present invention based simply on the teachings of WO96/014871.

Thus using the combination of amphiphiles disclosed in this invention, which may or may not be lamellar-forming, oil formulations can be constructed in mineral oil, triglycerides or squalene which will readily solubilise high concentrations of the macromolecules, and will retain these macromolecules even after dispersion of the oil phase in aqueous media. Although it is not a necessary condition of the invention, the mechanism by which macromolecules are incorporated into the final oil phase may, for example, be as a result of inclusion into reverse micelles.

In the first aspect the present invention provides a single phase hydrophobic preparation comprising a hydrophilic species, and an amphiphilic component comprising at least sodium docusate, a phospholipid and a nonionic amphiphile in an oil phase, wherein the moieties of the hydrophilic species are surrounded by the amphiphilic component with the hydrophilic head groups of the amphiphilic component orientated towards the hydrophilic species and wherein there is no chemical interaction, such as covalent interaction, between the amphiphilic component and the hydrophilic species; characterised in that said non-ionic amphiphile has a lipophilic chain comprising 10 to 20 carbons, and a head group comprising 2 to 10 oxyethylene groups or 1 to 3 hydroxyl groups. The molecules of the hydrophilic species are finely stably and homogeneously dispersed throughout the hydrophobic medium.

As used herein a “non-ionic amphiphile” is defined as an amphiphile which has a lipophilic chain and a head group as defined herein. The lipophilic chain of the non-ionic amphiphile comprises 10 to 20 carbons, preferably 12-18 carbons, more preferably 14-16 carbons. The lipophilic chain can comprise 10, 11, 12, 13, 14, 15, 16, 17, 18, 19 or 20 carbons. The head group comprises 2 to 10 oxyethylene groups or 1 to 3 hydroxyl groups. Preferably the head group comprises 4 to 8 oxyethylene groups. The head group can comprise 2, 3, 4, 5, 6, 7, 8, 9 or 10 oxyethylene groups. Alternatively the head group can comprise 1, 2 or 3 hydroxyl groups. Examples of non-ionic amphiphiles include polyoxethylene 2 hexadecyl ether (Brij 52), polyoxethylene 2 oleyl ether, polyoxyethylene 10 hexadecyl ether, polyoxyethylene 4 cetyl ether, polyoxethylene 4 myristyl ether, polyoxethylene 3 stearyl ether, polyoxethylene 4 lauryl ether, glycolic acid ethoxylate lauryl ether and lauryl sorbitan or mixtures thereof.

Suitable oil phases include hydrocarbons, e.g. non-polar oils such as vegetable oils including peanut oil, safflower oil, soya bean oil, cotton seed oil, corn oil, olive oil, almond oil, sesame oil, coconut oil, castor oil, chaulmoogra oil, persic oil, isopropyl myristate mineral oil including light paraffin, squalane and squalene, long chain fatty acids with unsaturated fatty acids such as oleic and linoleic acids being preferred, alcohols, particularly medium chain alcohols such as octanol and branched long chain alcohols such as phytol, isoprenoids, e.g. nerol, and geraniol, other alcohols such as t-butanol, terpineol, monoglycerides such as glycerol monooleate (GMO), other esters, e.g. ethyl acetate, amyl acetate and bornyl acetate, medium or long-chain mono-, di- or tri-glycerides and mixtures thereof, halogenated analogues of any of the above including halogenated oils, e.g. long chain fluorocarbons and iodinated triglycerides, e.g. lipidiol. Suitable triglycerides include those derived from the fractionated plant fatty acids or mixtures thereof. For examples mixtures of Caprylic, Capric, and Linoleic triglycerides such as Miglyol 818™ or mixtures of Propylene Glycol, Dicaprylate, and Dicaprate such as Miglyol 840™ can be used

Phospholipids having a phosphatidyl choline head group can be used and examples of such phospholipids include phosphatidyl choline (PC) itself, lyso-phosphatidyl choline (lyso-PC), soya-PC, sphingomyelin, derivatives of any of these, for example hexadecylphosphocholine or amphiphilic polymers containing phosphoryl choline and halogenated amphiphiles, e.g. fluorinated phospholipids. In the present application, the terms phosphatidyl choline (PC) and lecithin are used interchangeably. Suitable natural lecithins may be derived from any convenient source, for example egg and, in particular, soya.

In order for the amphiphilic components to be oriented with their headgroups directed towards the hydrophilic species, a method of preparation is required which causes the hydrophilic species to be surrounded by the amphiphiles before the oil phase is introduced, but after water has been removed. This can be achieved by creating a two-phase water-in-oil emulsion where the nonwater-miscible ‘oil phase’ is a hydrophobic phase that can readily be removed, e.g. by evaporation, or by lyophilisation. Lyophilisation is advantageous as a method for removing the oil phase, since the aqueous phase within the emulsion droplets can be removed at the same time.

It is very much preferred that the preparations of the invention are optically clear and this can be monitored by measuring turbidity at visible wave lengths and, in some cases, by checking for sedimentation over a period of time. Typically the optical density at 620 mm can be measured. A value of 0.2 or less, preferably 0.15 or less is considered to be clear.

In all of the structures of the present invention, the hydrophilic head groups of the amphiphile molecules face inwards towards the centre of the structure while the hydrophobic tails face outwards towards the solvent in which the hydrophobic species is dispersed.

Thus in a second aspect of this invention is provided a method of manufacture of a hydrophobic preparation containing a hydrophilic species which includes the following steps:
(i) Mixing a solution of sodium docusate, a phospholipid and a non-ionic amphiphile dissolved in a hydrophobic solvent with a solution of a hydrophilic species dissolved in an aqueous phase to form an emulsion,
(ii) removing the aqueous phase and hydrophobic solvent;
(iii) Adding an oil phase to the dry residue obtained in (ii).

As used herein the term “hydrophobic solvent” refers to a hydrophobic phase that can readily be removed, e.g. by evaporation, or by lyophilisation. Any volatile hydrophobic solvent with an appropriate melting point may be used. The solvent must be water immiscible and should be easily lyophilised, so it preferably has a freezing point between −10° C. and +15° C. Examples of suitable solvents include cyclohexane, cycloheptane and cyclooctane, mixtures of these compounds in a range of proportions, and mixtures with small proportions of tertiary-butanol added, sufficient to increase the solubility of the amphiphile to required levels. The hydrophobic solvent of choice will depend on the types of species to be solubilised and on the amphiphile. This can easily be determined by the person skilled in the art using the guidance found in the examples below.

The use of a phospholipid alone in this procedure does not lead to the production of a clear dispersion in a range of different oils. If a combination of the non-ionic amphiphile and sodium docusate is used, under conditions where free water is completely absent from the system, the results vary between a suspension of fine particles, or a fine homogenous but very turbid dispersion, depending on the ratio of amphiphilic substances. However, the inclusion of a phospholipid as a minor component with the sodium docusate and non-ionic amphiphile does give a clear dispersion. Where the oil phase is mineral oil, and the non-ionic amphiphile is POE 2 cetyl ether (Brij 52), optimal results are obtained when the weight ratio of components lies between 2:1:2 and 3:2:3 sodium docusate: phospholipid: non-ionic amphiphile. These ratios are indicative ratios only and, in particular, it should be pointed out that the precise ratios will depend on the nature of the oil and the amphiphile employed. Experiments can easily be conducted to determine the optimal ratios of the different components in any given case, as described in the examples given at the end of this specification.

Suitable aqueous phases include water, deuterium oxide and dimethyl sulphoxide (DMSO). Small quantities of additional hydrophilic agents may be admixed with the hydrophilic phase—eg glycol, glycerol, propylene glycol, propylene carbonate, PEG or mono or oligosaccharides.

The amphiphiles are dissolved in hydrophobic solvent at a level of up to 100 mg/ml total solute in solvent. The macromolecule, preferably an immunogen or immunomodulator, is dissolved in water or other suitable aqueous phase such as DMSO, usually at a concentration of 10-20 mg/ml. The aqueous phase is then added to the hydrophobic solvent, preferably in the ratio of 1:4 vol/vol, giving a homogenous dispersion after mixing by vortexing.

The average size of the emulsion particles will depend on the exact nature of both the hydrophobic and the aqueous phases. However, it may be in the region of 2 μm.

Dispersion of the aqueous phase in the hydrophobic solvent can be achieved by mixing, for example either by vigorous vortexing for a short time for example about 10 to 60 seconds, usually about 15 seconds, or by gentle mixing for several hours, for example using an orbital shaker.

The product of the process of the second aspect is new since it makes possible the production of a composition comprising a hydrophilic species which would not normally be soluble in a hydrophobic solvent, which is dissolved in oils such as mineral oil, squalane, squalene and triglycerides, wherein the hydrophilic species is retained to a high degree in the hydrophobic solvent after dispersion of the composition in aqueous phase. Other oils, which are normally solid at room temperature (eg tristearin, trilaurin, paraffin wax), can also be employed, if the step of addition of oil to the hydrophilic phase is performed at a temperature above the melting point of the oil concerned.

The compositions described above can be used to make a two phase composition. Thus in a third aspect the present invention provides a two phase composition comprising a hydrophilic phase and a hydrophobic phase, wherein said hydrophobic phase comprises a composition or preparation as described above. The two phase composition can be formed by contacting the composition or hydrophobic preparation with a hydrophilic phase such as an aqueous solution. Suitable hydrophilic phases comprise water, deuterium oxide and dimethyl sulphoxide (DMSO). Small quantities of additional hydrophilic agents may be admixed with the hydrophilic phase—eg glycol, glycerol, propylene glycol, propylene carbonate, PEG or mono or oligosaccharides.

In a preferred embodiment of the third aspect, the two-phase composition is an oil-in-water emulsion. Emulsions containing the hydrophobic preparations or compositions of the invention can also be used in the preparation of microcapsules. If the emulsion is formed from a gelatin-containing aqueous phase, the gelatin can be precipitated from the solution by coacervation by known methods and will form a film around the droplets of the hydrophile-containing hydrophobic phase. On removal of the hydrophilic phase, microcapsules will remain. This technology is known in the art, but is particularly useful in combination with the preparations of the present invention. Thus a fourth aspect the present invention provides a process for the preparation of an oil-in-water emulsion comprising the step of:

contacting a single phase hydrophobic preparation of the invention with a hydrophilic phase to form an oil-in-water emulsion.

In a preferred embodiment the hydrophilic phase comprises gelatin or albumin.

The oil-in-water double emulsions retain the hydrophilic solute within the hydrophobic oil phase with minimal leakage to the external aqueous compartment over varying periods of time. The oil used in this system is preferably mineral oil, squalane, squalene or triglyceride. Other oils, which are normally solid at room temperature (eg tristearin, trilaurin, paraffin wax), can also be employed, if the step of addition of oil to the hydrophilic phase is performed at a temperature above the melting point of the oil concerned.

If the outer hydrophilic phase is albumin, for example at a concentration of 50 mg/ml, or gelatin up to a level of 20% w/w, then retention is further enhanced. Thus the hydrophilic phase preferably comprises gelatin or albumin. The higher the degree of retention the more suitable the formulation is as a vaccine delivery vehicle.

The products of the present invention are extremely versatile and have many applications. In a fifth aspect the present invention provides a formulation comprising a preparation or composition of the invention and optionally one or more pharmaceutically acceptable excipients, diluents or carriers. Such formulations find use in medicine.

In one preferred embodiment of the invention the hydrophilic species in the composition or preparation is an immunogen. The formulation is preferably a vaccine. In a further aspect the present invention provides the use of a formulation of the invention as a vaccine.

In the present invention the term “hydrophilic species” relates to any species which is generally soluble in aqueous solvents but insoluble in hydrophobic solvents. The range of hydrophilic species of use in the present invention is diverse but hydrophilic macromolecules represent an example of a species that may be used.

A wide variety of macromolecules are suitable for use in the present invention. In general, the macromolecular compound will be hydrophilic or will at least have hydrophilic regions since there is usually little difficulty in solubilising a hydrophobic macromolecule in oily solutions. Examples of suitable macromolecules include proteins and glycoproteins, oligo and polynucleic acids, for example DNA and RNA, polysaccharides and supramolecular assemblies of any of these including, in some cases, whole cells or organelles. Examples of particular proteins which may be successfully solubilised by the method of the present invention include insulin, calcitonin, haemoglobin, cytochrome C, horseradish peroxidase, aprotinin, mushroom tyrosinase, erythropoietin, somatotropin, growth hormone, growth hormone releasing factor, galanin, urokinase, Factor IX, tissue plasminogen activator, superoxide dismutase, catalase, peroxidase, ferritin, interferon, Factor VIII and fragments thereof (all of the above proteins can be from any suitable source). Other macromolecules may be used are FITC-labelled dextran and RNA extract from Torulla yeast. In particular the macromolecule can be a collagen such as collagen type I or collagen type II. A formulation containing collagen type II is a promising candidate for oral down-regulation of immune responses in rheumatoid arthritis. The macromolecule can also be an immunogen, especially for use in a vaccine composition. Usually a minimum concentration of 2.5 mg macromolecule is incorporated into 1 ml of oil, so that the concentration of the macromolecule in the initial hydrophilic solvent is at least 10 mg/ml.

As used herein, the term “immunogen” relates to a species capable of eliciting an immune outcome. This outcome can be a typical immune response, e.g. the production of antibodies, or the triggering of differentiation or expansion of specific populations of T cells, and can be systemic or local, e.g. restricted to a mucosal response. Alternatively, the immune outcome can be, for instance immune tolerance, in which the naïve immune system is rendered unresponsive to challenge by a specific antigen. Another alternative outcome may be desensitization, in which a pre-existing tendency to an autoimmune or allergic response (IgE) against a specific antigen is reduced.

The immunogen may be selected from, but not limited to, Diphtheria toxoid, tetanus toxoid, botulin toxoid, snake venom antigens, viral antigens e.g. Hepatitis virus A, B, C, D, or E antigens, whooping cough subunit, influenza A and/or B (either whole-killed, virus or protein subunits), H1N1 swine flu, H5N1 bird flu, polio virus, rotavirus, mumps, measles virus, chickenpox, meningitis, rubella, respiratory syncitial virus, HIV, EV71, dengue virus antigens, yellow fever antigens, human papilloma virus antigens, herpes virus HSV 1 or HSV2 antigens, ebola virus, porcine reproductive and respiratory syndrome virus, porcine circovirus type 2, West Nile virus, Japanese Encephalitis virus, hand-foot-and-mouth disease antigens, whole bacteria or extracts thereof e.g. BCG, other mycobacterial antigens, enteric disease pathogens and antigens thereof including for example cholera antigens, salmonella species, eschericia species, Helicobacter pylori antigens, P. aeruginosa, chlamydia species, neisseria species, yersinia species, fungi or fungal antigens, H. influenzae A or B (with or without carrier protein), protozoal antigens, e.g. malaria, leishmania, toxoplasma, trypanosoma, trematode antigens, e.g. schistosoma, cestode antigens e.g. from cysticerca, echinococcus, nematode antigens e.g. toxocara, hookworm and filarial, spirochete antigens e.g. borrelia species, surface membrane epitopes specific for cancer cells, and cell receptor targeting anti-inflammatory modulators, polymer conjugates of steroids. Immunogens for use in down-regulating immune responses include HLA antigens, pollens, dust mite antigens, bee stings or food allergens such as gluten or peanuts, glutamic acid dehydrogenase, insulin, or conjugates containing insulin subcomponents, for treatment of diabetes. In addition, the immunogen can be a collagen such as collagen type I or collagen type II. A formulation containing collagen type II is a promising candidate for oral down-regulation of immune responses in rheumatoid arthritis. The immunogens can be peptides, proteins, lipids, sugars, nucleic acids, steroids and/or conjugates of one or more of these agents in combination. It is also possible, where the antigen is a peptide, polysaccharide or other antigen, to conjugate it with at least one medium- or long-chain hydrocarbon tail.

One advantage of the present invention is that different antigens (e.g. proteins and polysaccharides) can be co-presented together in the same vehicle to elicit an enhanced immune response by virtue of one component acting as a carrier for the other, without the need for any covalent linkage.

In cases where an up-regulation of the immune response is desired, the immunogen may be combined with one or more other molecules (immunostimulants or adjuvants), co-entrapped within the same oil phase as the immunogen. Such adjuvants may include cholera toxin B fragment and analogues and derivatives thereof, E. coli heat labile toxin and analogues and derivatives thereof, BCG, CpG-containing oligonucleotide sequences, tetanus toxoid, diphtheria toxoid, bacterial lipid A (intact or detoxified), monophosphoryl lipid A.

In another embodiment the immunogen is co-solubulised with one or more cytokines in order to enhance the response. Examples of suitable cytokines include IL-4, IL-10, IL-12, and γ-interferons. Other immunostimulants may also be incorporated, for example monophosphoryl lipid A, mycobacterial extracts, muramyl dipeptide and analogues, tuftsin and cholera subunit B and heat labile toxin of E. coli.

It may also be convenient to co-solubilise a small molecule such as a vitamin in association with a macromolecule, particularly a polysaccharide such as a cyclodextrin. Small molecules such as vitamin B12 may also be chemically conjugated with macromolecules and may thus be included in the compositions.

The process of the present invention allows encapsulation at a much lower amphiphile: protein ratio, as compared to the methods in the prior art, such as WO95/13795. This allows more of the macromolecule to be incorporated into oils such as triglycerides and mineral oil. In addition the retention of the macromolecules in the oil after dispersion in the aqueous medium is higher.

In addition to macromolecules, the processes of the present invention are of use in solubilising smaller organic molecules. Examples of small organic molecules include glucose, carboxyfluorescein and many pharmaceutical agents, for example anti-cancer agents, but, of course, the process could equally be applied to other small organic molecules, for example vitamins or pharmaceutically or biologically active agents. In addition, compounds such as calcium chloride and sodium phosphate can also be solubilised using this process. Indeed, the present invention would be particularly advantageous for pharmaceutically and biologically active agents since the use of non aqueous solutions may enable the route by which the molecule enters the body to be varied, for example to increase bioavailability.

Another type of species that may be included in the hydrophobic compositions of the invention is an inorganic material such as a small inorganic molecule or a colloidal substance, for example a colloidal metal. The process of the present invention enables some of the properties of a colloidal metal such as colloidal gold, palladium, platinum or rhodium, to be retained even in hydrophobic solvents in which the particles would, under normal circumstances, aggregate. This could be particularly useful for catalysis of reactions carried out in organic solvents.

Other large particulate materials can also be encapsulated using this method, for example viruses and bacteria, either live, attenuated or inactivated.

In other aspects the invention provides:

A cosmetic formulation comprising the preparation or composition of the invention and optionally one or more excipients, diluents or carriers.

A method of treating a subject comprising administering the preparation or composition of the invention

The preparation or composition of the invention comprising collagen or fragments thereof for use in treating rheumatoid arthritis. The collagen is preferably collagen type II.

One way in which the compositions of the present invention may be used is for the oral delivery to mammals, including man, of substances, which would not, under normal circumstances, be soluble in lipophilic solvents. This may be of use for the delivery of dietary supplements such as vitamins or for the delivery of biologically active substances, particularly proteins or glycoproteins, including insulin growth hormones and immunogens.

In a further application, it is possible to encapsulate or microencapsulate, for example by the method described above, nutrients such as vitamins which can then be used, not only as human food supplements but also in agriculture and aquaculture, one example of the latter being in the production of a food stuff for the culture of larval shrimps.

In addition, the compositions find application in the preparation of pharmaceutical or other formulations for parenteral administration, as well as formulations for topical or ophthalmic use. For this application, it is often preferable to use an emulsion of the oil solution and an aqueous phase as described above.

Many therapeutic and prophylactic treatments are intended for sustained or delayed release or involve a two component system, for example including a component for immediate release together with a component for delayed or sustained release. Because of their high stability, the preparations of the invention are particularly useful for the formulation of a macromolecule intended for sustained or delayed release.

The longer shelf life of the compositions of the present invention is a particular advantage in the pharmaceutical area.

The hydrophile-in-oil preparations may find application in the pharmaceutical or similar industries for flavour masking. This is a particular problem in the pharmaceutical industry since many drugs have unpleasant flavours and are thus unpopular with patients, especially children.

A further use is in the cosmetics industry where, again, hydrophobic preparations of hydrophilic compounds can very easily be incorporated into a cosmetic formulation. Examples of macromolecules that may be used in this way include those with moisturizing or enzymatic action of some sort. The invention can also be used for the incorporation of proteins such as collagen into dermatological creams and lotions.

The formulations of this invention may be presented in conjunction with other agents to allow its administration to humans and animals for therapeutic and other purposes. The resulting compositions may comprise a paste, a cream, a gel, a semi-solid or a two-phase solid dispersion. The formulations may be applied topically, orally, optically, nasally or as a suppository, or may be administered as an injection (eg intra-muscular, subcutaneous or intra-dermal). In the case where oral administration is effected, the composition may be in the form of a liquid, and lozenge, a gel, or admixed with a dry powder, any of these forms being ingested either in free form, or encapsulated, for example in a gelatin, starch or HPMC hard capsule shell, on in a soft capsule such as a soft gelatin capsule. Optionally, these capsules may be enteric-coated to allow them to pass through the stomach without interacting with stomach contents, but subsequently dissolving in the small or large intestine. Suitable coatings are know in the art. Alternatively, for suitable routes of administration, e.g. nasal, pulmonary, buccal and sub-lingual administration, the composition can be in the form of an aerosol. The aerosol can be formed of either oil droplets or oil-in-water droplets containing the hydrophobic preparation of the application.

The compositions of the invention may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; liquid emulsions.

Pharmaceutical formulations adapted for topical administration may be formulated as ointments, creams, suspensions, lotions, powders, solutions, pastes, gels, sprays, aerosols or oils.

For applications to the eye or other external tissues, for example the mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the microemulsions comprising the active ingredient.

Pharmaceutical formulations adapted for administration by inhalation include fine particle dusts or mists which may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may also include other agents conventional in the art having regard to the type of formulation in question, for example those suitable for oral administration may include flavouring agents.

Finally, the invention has numerous uses in the field of chemical and biological synthesis, for example, non-aqueous enzymatic synthesis.

The invention will now be further described with reference to the following non-limiting examples and the following figures:

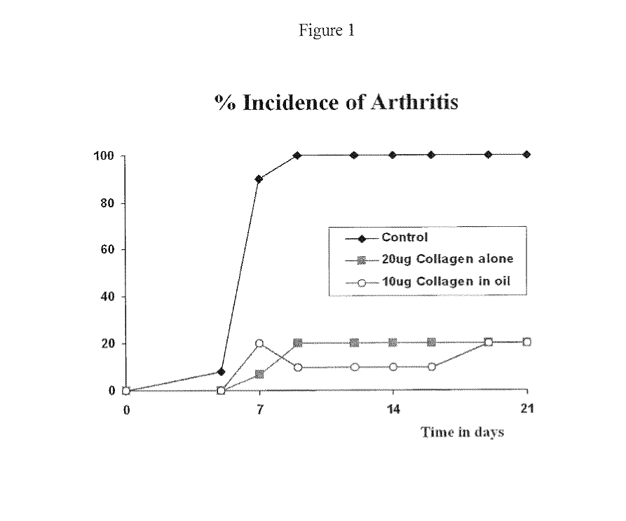

FIG. 1 shows the incidence of arthritis in mice following treatment with the preparation of the inventions comprising collagen.

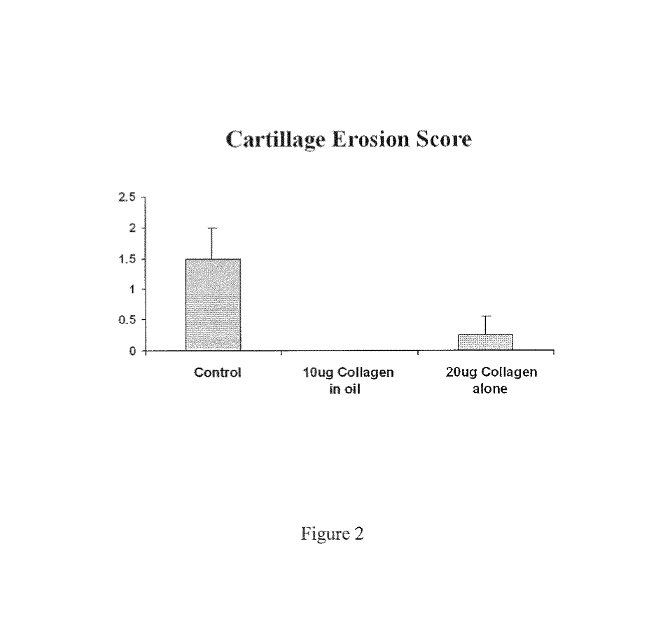

FIG. 2 shows the effect of administration of the preparation of the invention comprising collagen on cartilage erosion in arthritic mice.

### EXAMPLE 1

### Formation of Reverse Micelles in Mineral Oil with a Combination of Docusate, Brij 52 and Soya PC Containing Aprotinin

1. Into an 8 ml glass screw-capped vial 300 mg of sodium docusate, was weighed out, and 2.7 ml of cyclohexane added. After screwing the cap on tightly the vial was shaken with warming until the contents were dissolved, to give a solution with a concentration close to 100 mg/ml.
2. Into an 8 ml glass screw-capped vial 300 mg of Brij 52 (polyoxyethylene 2 cetyl ether) was weighed out, and 2.7 ml of cyclohexane added. After screwing the cap on tightly the vial was shaken with warming until the contents were dissolved, to give a solution with a concentration close to 100 mg/ml.
3. Into an 8 ml glass screw-capped vial 300 mg of Soya phosphatidylcholine was weighed out, and 2.7 ml of cyclohexane added. After screwing the cap on tightly the vial was shaken with warming until the contents were dissolved, to give a solution with a concentration close to 100 mg/ml.
4. 10×2 ml glass screw-capped vials were labelled 1 to 10, and into each tube solutions from steps 1, 2 and 3 were dispensed in the volumes indicated in the table below.

| | Docusate | Brij 52 | Soya |
|---|---|---|---|
| | solution | solution | phosphatidylcholine |
| Tube No: | ul | ul | ul |
| 1 | 0 | 400 | — |
| 2 | 100 | 300 | — |
| 3 | 200 | 200 | — |
| 4 | 300 | 100 | — |
| 5 | 400 | 0 | — |
| 6 | 0 | 400 | 100 |
| 7 | 100 | 300 | 100 |
| 8 | 200 | 200 | 100 |
| 9 | 300 | 100 | 100 |
| 10 | 400 | 0 | 100 |

5 To each sample 100 ul of aprotinin solution at 20 mg/ml was added, vortexed briefly and then frozen rapidly in a glycerol bath and then exposed to a vacuum of 1 mbar of less, overnight, to remove cyclohexane and water.
6 The following day 450 ul of Mineral oil was added to each of the vials, which were then capped and placed on a roller mixer until a single homogenous phase was obtained.

200 ul of each of the dispersions transferred to a separate well of a microplate, and the scattering is evaluated by measuring optical density at 620 nm in a microplate reader.

As can be seen from the table below, most effective dissolution was observed (as judged be absence of scattering, indicated by a reduction in the OD) when a combination of two or more amphiphiles was employed to form the hydrophobic phase.

| Samples | Absorbance | Samples with | Absorbance |
|---|---|---|---|
| without PC | readings | PC | readings |
| 1 | 0.514 | 6 | 1.041 |
| 2 | 0.34 | 7 | 1.22 |
| 3 | 0.2 | 8 | 0.089 |
| 4 | 0.231 | 9 | 0.076 |
| 5 | 0.928 | 10 | 0.49 |

### EXAMPLE 2

### Leakage of Aprotinin from Reverse Micelles in Mineral Oil with a Combination of Docusate, Brij 52 and Soya PC

1. In 2 ml vials 400 ul of PBS and 20 ul of the aprotinin oil formulation from Example 1 were added. The samples were mixed vigorously till dispersed and then span down in the centrifuge at 3000 rpm for 10 minutes.
2. 50 ul of the aqueous phase of each sample was transferred to a separate well of a microplate, to which 150 ul of Bradford protein reagent was added. Protein concentration was determined by measuring optical density at 570 nm in a microplate reader and comparing with a standard curve. Leakage was inferred by comparison with a control containing aprotinin alone.
As can be seen from the table below, leakage was minimal when a combination of two or more amphiphiles was employed to form the hydrophobic phase.

| Samples | | Samples with | |
|---|---|---|---|
| without PC | % Leakage | PC | % Leakage |
| 1 | 12.42 | 6 | 1.86 |
| 2 | 0.38 | 7 | 0.90 |
| 3 | 0.27 | 8 | 0.39 |
| 4 | 0.27 | 9 | 0.25 |
| 5 | 0.81 | 10 | 0.42 |

### EXAMPLE 3

### Formation of Reverse Micelles in Mineral Oil with a Combination of Docusate, Brij 52 and Soya PC Containing the Macromolecular Polymeric Dye Poly-R478.

1 Into an 8 ml glass screw-capped vial 300 mg of sodium docusate, was weighed out, and 2.7 ml of cyclohexane added. After screwing the cap on tightly the vial was shaken with warming until the contents were dissolved, to give a solution with a concentration close to 100 mg/ml.
2 Into an 8 ml glass screw-capped vial 300 mg of Brij 52 (polyoxyethylene 2 cetyl ether) was weighed out, and 2.7 ml of cyclohexane added. After screwing the cap on tightly the vial was shaken with warming until the contents were dissolved, to give a solution with a concentration close to 100 mg/ml.
3 Into an 8 ml glass screw-capped vial 300 mg of Soya phosphatidylcholine was weighed out, and 2.7 ml of cyclohexane added. After screwing the cap on tightly the vial was shaken with warming until the contents were dissolved, to give a solution with a concentration close to 100 mg/ml.
4 10×2 ml glass screw-capped vials were labelled 1 to 10, and into each tube solutions from steps 1, 2 and 3 were dispensed in the volumes indicated in the table below.

| | Docusate | Brij 52 | Soya |
|---|---|---|---|
| | solution | solution | phosphatidylcholine |
| Tube No: | ul | ul | ul |
| 1 | 0 | 400 | — |
| 2 | 100 | 300 | — |
| 3 | 200 | 200 | — |
| 4 | 300 | 100 | — |
| 5 | 400 | 0 | — |
| 6 | 0 | 400 | 100 |
| 7 | 100 | 300 | 100 |
| 8 | 200 | 200 | 100 |
| 9 | 300 | 100 | 100 |
| 10 | 400 | 0 | 100 |

5 To each sample 100 ul of poly-R478 solution at 20 mg/ml was added, vortexed briefly and then frozen rapidly in a glycerol bath and then exposed to a vacuum of 1 mbar of less, overnight, to remove cyclohexane and water.
6 The following day 450 ul of Mineral oil was added to each of the vials, which were then capped and placed on a roller mixer until a single homogenous phase was obtained.
7 200 ul of each of the dispersions transferred to a separate well of a microplate, and the scattering is evaluated by measuring the difference in optical density between 620 and 492 nm in a microplate reader.
As can be seen from the table below, most effective dissolution was observed (as judged be absence of scattering indicated by a reduction in the OD) when a combination of two or more amphiphiles was employed to form the hydrophobic phase.

| Samples | Absorbance | Samples with | Absorbance |
|---|---|---|---|
| without PC | readings | PC | readings |
| 1 | 0.87 | 6 | 0.129 |
| 2 | 0.27 | 7 | 0.043 |
| 3 | 0.208 | 8 | 0.078 |
| 4 | 0.485 | 9 | 0.147 |
| 5 | 0.561 | 10 | 0.416 |

### EXAMPLE 4

### Leakage of the Macromolecular Dye Poly-R478 from Reverse Micelles in Mineral Oil with a Combination of Docusate, Brij 52 and Soya PC

1 In 2 ml vials 400 ul of PBS and 20 ul of the poly-R478 oil formulation from Example 1 were added. The samples were mixed vigorously till dispersed and then span down in the centrifuge at 3000 rpm for 10 minutes.
2 200 ul of the aqueous phase of each sample was transferred to a separate well of a microplate, and the concentration of the dye was determined by measuring optical density at 492 nm in a microplate reader and comparing with a standard curve. Leakage was inferred by comparison with a control containing poly-R478 alone.
As can be seen from the table below, leakage was minimal when a combination of two or more amphiphiles was employed to form the hydrophobic phase.

| Samples | | Samples with | |
|---|---|---|---|
| without PC | % Leakage | PC | % Leakage |
| 1 | 100 | 6 | 15 |
| 2 | 32 | 7 | 4 |
| 3 | 25 | 8 | 8 |
| 4 | 59 | 9 | 17 |
| 5 | 68 | 10 | 50 |

### EXAMPLE 5

### Leakage of Lysozyme from Reverse Micelles in Different Oils with a Combination of Sodium Docusate, Brij 52 and Soya Phosphatidyl Choline, Compared with Soya Phosphatidyl Choline Alone

Hydrophobic preparations employing soya phosphatidyl choline as amphiphile were constructed as follows, prior to addition of the oil phase:
1. Soya phosphatidylcholine (SPC) was added to distilled water in a 20 ml vial (1 g of SPC+9 ml water), and the mixture was then vortexed until dispersed completely.
2. The dispersion was then extruded twice through 0.2 um Anatop filters.
3. In one 8 ml vial 20 mg of lysozyme was weighed out and dissolved in 4 ml of the liposome dispersion from step 2 above.
4. 6×400 ul aliquots of the lysozyme solution from step 3 were transferred to fresh glass screw-capped 2 ml vials, then frozen rapidly in the glycerol and maintained at −30 degC for one hour.
5. The vials were then lyophilised over night.

Hydrophobic preparations employing sodium docusate, Brij 52 and soya phosphatidyl choline as amphiphile were constructed as follows, prior to addition of the oil phase:
1. Soya phosphatidylcholine (SPC) was dissolved in cyclohexane at a concentration of 100 mg/ml in an 8 ml vial. (600 mg of SPC+5.4 ml cyclohexane).
2. Sodium docusate was dissolved in cyclohexane at a concentration of 100 mg/ml in an 8 ml vial. (600 mg of docusate+5.4 ml cyclohexane)
3. Brij 52 was dissolved in cyclohexane at a concentration of 100 mg/ml in an 8 ml vial (600 mg of Brij 52+5.4 ml cyclohexane)
4. Lysozome was dissolved in distilled water at 20 mg/ml in an 8 ml vial.
5. The SPC, docusate and Brij 52 solutions were mixed at the ratio (2:3:3) in 20 ml vial by adding 2 ml, 3 ml and 3 ml of each of the respective solutions, and mixing well.
6. To 6×2 ml glass screw-capped vials was added 400 ul of the SPC:Brij52:Docusate/cyclohexane solution.
7. 100 ul of lysozyme solution was added to each of the vials in the previous step while vortexing (aprox. 10 sec.) The dispersions were frozen immediately in a −20 degC glycerol bath and then incubated in the −30 degC for approximately 1 hour.
8. The samples were then lyophilised over night.
Oil phases were prepared from the dried residues obtained above as follows:
9. When the samples were dried, 360 ul of different oils listed below were added to each vial. The vials were then capped and placed on a roller mixer until homogenous oil phases were obtained.

| Sample | Oil added |
|---|---|
| 1 | Mineral oil |
| 2 | Squalene |
| 3 | Glycerol monooleate |
| 4 | Miglyol ™ 840 |
| 5 | Miglyol ™ 818 |
| 6 | Medium-chain Monoglyceride |

Leakage of protein from the hydrophobic phases after dispersion in aqueous phase was quantified as follows:
1. Fluorescamine was dissolved in acetone at 0.2 mg.m1 (2 mg of Fluorescamine+10 ml of Acetone).
2. Into 12×8 ml labelled vials 1.5 ml of PBS buffer and 75 ul of each hydrophobic phase were introduced. The samples were then shaken vigorously and spun down for 50 minutes at 1000 g.
3. 1 ml of the aqueous phase from each sample was then transferred to fresh vials and vortexed with 50 ul of fluorescamine/acetone solution for 10 seconds.
4. 200 ul of each sample transferred onto a white microplate and the fluorescence was measured at Ex=390 nm; Em=465 nm; Cut off=455 nm in a Molecular Devices Spectramax fluorescence plate reader.
5. A standard curve was prepared using a range of concentrations of free lysozyme, and the leakage of protein from each oil phase was calculated as a percentage of the original quantity incorporated.

| | | Leakage from |
|---|---|---|
| | Leakage from | hydrophobic phases |
| | hydrophobic phases | containing a |
| | containing Soya PC | combination of |
| Oil phase | alone (%) | amphiphiles (%) |
| Mineral oil | 100 | 0.0 |
| Squalene | 54 | 0.1 |
| Glycerol monooleate | 5 | 0.1 |
| Miglyol 840 | 13 | 0.1 |
| Miglyol 818 | 50 | 3.8 |
| Medium-chain | 40 | 0.6 |
| Monoglyceride | | |

As can be seen from the table, for a range of different oils, leakage of protein from the oil is very much reduced when a combination of amphiphiles is employed, in contrast to phosphatidyl choline alone.

### EXAMPLE 6

### Formation of Reverse Micelles in Mineral Oil Using a Range of Different Amphiphiles in combination with Sodium Docusate and Soya Phosphatidyl Choline

1 Into an 8 ml glass screw-capped vial 300 mg of sodium docusate, was weighed out, and 2.7 ml of cyclohexane added. After screwing the cap on tightly the vial was shaken with warming until the contents were dissolved, to give a solution with a concentration close to 100 mg/ml.
3 Into an 8 ml glass screw-capped vial 300 mg of each of the amphiphiles described in the table below was weighed out, and 2.7 ml of cyclohexane added. After screwing the cap on tightly the vial was shaken with warming until the contents were dissolved, to give a solution with a concentration close to 100 mg/ml.
4 Into an 8 ml glass screw-capped vial 300 mg of Soya phosphatidylcholine was weighed out, and 2.7 ml of cyclohexane added. After screwing the cap on tightly the vial was shaken with warming until the contents were dissolved, to give a solution with a concentration close to 100 mg/ml.
5 10×2 ml glass screw-capped vials were labelled 1 to 10, and into each tube solutions from steps 1, 2 and 3 were dispensed in the volumes indicated in the table below.

| | Docusate | Amphiphile | Soya |
|---|---|---|---|
| | solution | solution | phosphatidylcholine |
| TubeNo: | ul | ul | ul |
| 1 | 0 | 400 | — |
| 2 | 100 | 300 | — |
| 3 | 200 | 200 | — |
| 4 | 300 | 100 | — |
| 5 | 400 | 0 | — |
| 6 | 0 | 400 | 100 |
| 7 | 100 | 300 | 100 |
| 8 | 200 | 200 | 100 |
| 9 | 300 | 100 | 100 |
| 10 | 400 | 0 | 100 |

5 To each sample 100 ul of lysozyme solution at 20 mg/ml was added, vortexed briefly and then frozen rapidly in a glycerol bath and then exposed to a vacuum of 1 mbar of less, overnight, to remove cyclohexane and water.
6 The following day 450 ul of Mineral oil was added to each of the vials, which were then capped and placed on a roller mixer until a single homogenous phase was obtained.
7 200 ul of each of the dispersions transferred to a separate well of a microplate, and the scattering is evaluated by measuring the difference in optical density between 620 and 492 nm in a microplate reader.
As can be seen from the tables below, most effective dissolution was observed (as judged by absence of scattering indicated by a reduction in the OD) when a combination of two or more amphiphiles was employed to form the hydrophobic phase.

Polyoxyethylene 10 Hexadecyl

Ether

| Sample No. | Without PC | Sample No. | With PC |
|---|---|---|---|
| 1 | 2.179 | 6 | 2.148 |
| 2 | 1.377 | 7 | 1.09 |
| 3 | 0.142 | 8 | 0.156 |
| 4 | 0.08 | 9 | 0.091 |
| 5 | 0.359 | 10 | 0.101 |

Polyoxyethylene 2 Stearyl Ether

| Sample No. | Without PC | Sample No. | With PC |
|---|---|---|---|
| 1 | 1.693 | 6 | 1.716 |
| 2 | 0.124 | 7 | 0.105 |
| 3 | 0.096 | 8 | 0.099 |
| 4 | 0.085 | 9 | 0.094 |
| 5 | 0.462 | 10 | 0.101 |

Polyoxyethylene 4 Cetyl Ether

| Sample No. | Without PC | Sample No. | With PC |
|---|---|---|---|
| 1 | 0.604 | 6 | 0.693 |
| 2 | 0.096 | 7 | 0.085 |
| 3 | 0.088 | 8 | 0.096 |
| 4 | 0.087 | 9 | 0.084 |
| 5 | 0.482 | 10 | 0.403 |

Polyoxyethylene 4 Myristyl Ether

| Sample No. | Without PC | Sample No. | With PC |
|---|---|---|---|
| 1 | 0.76 | 6 | 0.546 |
| 2 | 0.099 | 7 | 0.091 |
| 3 | 0.104 | 8 | 0.102 |
| 4 | 0.082 | 9 | 0.086 |
| 5 | 0.378 | 10 | 0.106 |

Polyoxyethylene 3 Stearyl Ether

| Sample No. | Without PC | Sample No. | With PC |
|---|---|---|---|
| 1 | 2.011 | 6 | 2.476 |
| 2 | 1.665 | 7 | 1.474 |
| 3 | 0.093 | 8 | 0.474 |
| 4 | 0.081 | 9 | 0.087 |
| 5 | 0.466 | 10 | 0.097 |

Polyoxyethylene 4 Lauryl Ether

| Sample No. | Without PC | Sample No. | With PC |
|---|---|---|---|
| 1 | 0.401 | 6 | 0.172 |
| 2 | 0.106 | 7 | 0.093 |
| 3 | 0.089 | 8 | 0.097 |
| 4 | 0.098 | 9 | 0.079 |
| 5 | 0.185 | 10 | 0.107 |

Glycolic Acid Ethoxylate Lauryl Ether

| Sample No. | Without PC | Sample No. | With PC |
|---|---|---|---|
| 1 | 0.295 | 6 | 0.297 |
| 2 | 0.584 | 7 | 0.107 |
| 3 | 0.091 | 8 | 0.101 |
| 4 | 0.103 | 9 | 0.082 |
| 5 | 0.202 | 10 | 0.115 |

Polyoxyethylene 2 Oleyl Ether

| Sample No. | Without PC | Sample No. | With PC |
|---|---|---|---|
| 1 | 0.292 | 6 | 0.64 |
| 2 | 0.112 | 7 | 0.092 |
| 3 | 0.097 | 8 | 0.1 |
| 4 | 0.096 | 9 | 0.08 |
| 5 | 0.247 | 10 | 0.105 |

Lauryl Sorbitan

| Sample No. | Without PC | Sample No. | With PC |
|---|---|---|---|
| 1 | 0.396 | 6 | 0.106 |
| 2 | 0.104 | 7 | 0.091 |
| 3 | 0.092 | 8 | 0.098 |
| 4 | 0.095 | 9 | 0.08 |
| 5 | 0.169 | 10 | 0.105 |

### EXAMPLE 7

### Incorporation of Lysozyme into Reverse Micelles in Mineral Oil at Different Protein Concentrations with a Combination of Sodium Docusate, Brij 52 and Soya Phosphatidyl Choline, Compared with Soya Phosphatidyl Choline Alone

1. Hydrophobic preparations employing soya phosphatidyl choline as amphiphile were constructed as described in Example 5, except that the quantities of lysozyme were adjusted in order to achieve final concentrations of protein in mineral oil between 50 and 0 mg/ml, as shown in the table below. The concentration of lipid was 100 mg/ml.
2. Hydrophobic preparations employing sodium docusate, Brij 52 and soya phosphatidyl choline as amphiphile were constructed as described in Example 5, except that the quantities of lysozyme were adjusted in order to achieve final concentrations of protein in mineral oil between 50 and 0 mg/ml, as shown in the table below. The concentration of lipid was 100 mg/ml.
3. After addition of mineral oil, all the samples were mixed on a roller mixer for two hours, then 100 ul of each sample was transferred to a separate well of a clear microplate, and the optical density measured at 620 nm. Results are shown in the table below.

| Lysozyme concentration | Soya PC | Amphiphile mixture |
|---|---|---|
| mg/ml of oil | OD 620 nm | OD 620 nm |
| 50 | 1.669 | 1.514 |
| 37.5 | 1.179 | 0.616 |
| 25 | 1.169 | 0.071 |
| 18.75 | 0.752 | 0.061 |
| 12.5 | 0.511 | 0.066 |
| 6.25 | 0.414 | 0.055 |
| 3.125 | 0.125 | 0.059 |
| 0 | 0.09 | 0.071 |

As can be seen, clear solutions of protein in oil indicated by an OD reading less than 0.2 were achieved up to a concentration of 25 mg/ml for the amphiphile mixture, while dissolution in the formulations containing Soya PC alone could only be achieved with protein at a concentration of 3.125 mg/ml or below. Thus, the method described in the present invention is far superior to that described in the prior art (eg WO96/014871), in terms of quantity of protein which can be incorporated into oil.

### EXAMPLE 8

### Incorporation of Lysozyme into Reverse Micelles in Mineral Oil Containing Different Levels of Amphiphile, Using a Combination of Sodium Docusate, Brij 52 And Soya Phosphatidyl Choline, Compared with Soya Phosphatidyl Choline Alone

1. Hydrophobic preparations employing soya phosphatidyl choline as amphiphile were constructed as described in Example 5, except that the quantities of soya lecithin were adjusted in order to achieve final concentrations of lipid in the oil of 100, 87.5, 75, 62.5, 50, 37.5, 25, 12.5, 6.25, 2.5 and 0 mg/ml. After drying down of the lipid residues, sufficient mineral oil was added to achieve a final volume of 400 ul (assuming a density of amphiphile of 1 g/ml). The final concentration of lysozyme in the oil was 1 mg/ml.
2. Hydrophobic preparations employing sodium docusate, Brij 52 and soya phosphatidyl choline as amphiphile were constructed as described in Example 5, except that the quantities of soya lecithin were adjusted in order to achieve final concentrations of lipid in the oil of 100, 87.5, 75, 62.5, 50, 37.5, 25, 12.5, 6.25, 2.5 and 0 mg/ml. After drying down of the lipid residues, sufficient mineral oil was added to achieve a final volume of 400 ul (assuming a density of amphiphile of 1 g/ml). The final concentration of lysozyme in the oil was 1 mg/ml.
3. After addition of mineral oil, all the samples were mixed on a roller mixer for two hours, then 100 ul of each sample was transferred to a separate well of a clear microplate, and the optical density measured at 620 nm. Results are shown in the table below

| Amphiphile | | |
|---|---|---|
| concentration | Soya PC | Amphiphile mixture |
| (mg/ml of oil) | OD 620 nm | OD 620 nm |
| 100 | 0.171 | 0.087 |
| 87.5 | 0.274 | 0.097 |
| 75 | 0.233 | 0.09 |
| 62.5 | 0.646 | 0.085 |
| 50 | 0.592 | 0.08 |
| 37.5 | 0.455 | 0.083 |
| 25 | 0.332 | 0.087 |
| 12.5 | 0.351 | 0.104 |
| 6.25 | 0.404 | 0.098 |
| 2.5 | 0.426 | 0.216 |
| 0 | 0.415 | 0.3 |

As can be seen, clear solutions of protein in oil indicated by an OD less than 0.2 were achieved down to a concentration of 6.25 mg/ml for the amphiphile mixture (amphiphile:protein ration 6.25:1 wt/wt), while dissolution in the formulations containing Soya PC alone could only be achieved with amphiphile at a concentration of 100 mg/ml or above (amphiphile:protein ratio 100:1 wt/wt). Thus, the method described in the present invention is far superior to that described in the prior art (eg WO96/014871) in terms of economy of requirement for amphiphile.

### EXAMPLE 9

### Manufacture of Vaccine Formulation Containing Collagen Type II

1. Into one 8 ml glass screw-capped vial 0.5 ml of glacial acetic acid and 4.5 ml of dimethyl sulfoxide (DMSO) were added and mixed well by shaking 1 ml of this mixture was added to 5 mg of collagen type II, and then left overnight with gentle mixing at room temperature to dissolve.
2. Into 3×8 ml glass screw-capped vial 200 mg each of sodium docusate, soy phosphatidyl choline and Brij 52 were weighed out and dissolved in 1.8 ml of cyclohexane, with warming. Into one 8 ml vial 1.5 ml of docusate solution, 1.5 ml of Brij 52 solution and 1 ml of phospholipid solution were dispensed and mixed well.
3. 1 ml of amphiphile solution in cyclohexane was transferred to a fresh 8 ml glass vial, and 0.2 ml of collagen solution from step 1 was added, vortexed rapidly for 30 seconds, then frozen rapidly with shaking in a glycerol/water −30° C. cooling bath. The vial was allowed to stand in ice for five minutes and then transferred to a −30° C. freezer for twenty minutes.
4. The contents of the vial were lyophilized overnight by exposing to a vacuum of 1 mbar at +4° C. on a Genevac vacuum pump. After drying, 900 ul of mineral oil was added to the vial contents and shaken gently until all the contents had dissolved. The concentration of collagen in the oil is 1 mg per ml.
5. In a 200 ml glass conical flask 20 g of gelatin was weighed out, and 80 g of distilled water was added with shaking. The mixture was then heated on a magnetic stirrer at 50° C. until all the protein had dissolved.
6. 3 ml of gelatin solution was transferred to 6 pre-warmed 8 ml glass vial in a 37° C. water bath.
7 To each vial, 120 ul of oil from step 4 (containing 120 ug collagen) were added and mixed gently by slow vortexing for ten seconds. The vials were allowed to stand at room temperature until the contents had solidified. After flushing with nitrogen and capping the vials, they were stored at +4° C. until required for further use. A dose of 10 ug of collagen is contained in approximately 0.25 ml of gelatin solution.

### EXAMPLE 10

### Example of Use of Vaccine Preparation Containing Collagen Administered Orally to Down-Regulate Severity of Rheumatoid Arthritis in a Mouse Model

1. Ten-twelve week old male DBA-1 mice were weighed and divided into 3 groups (n=8/group) as outlined below. All groups were treated orally (by gavage) 4 times (days −10, −7, −5 & −3) prior to induction of collagen-induced arthritis (CIA) by injection of 100 ug collagen in Complete Freunds Adjuvant at the base of the tail.
2. Arthritis was induced in all animals on day 0, followed by a boost on day 21, and incidence and severity assessed and scored on days 5, 7, 9, 12, 14, 16, 19 and 21 after the boost. Three treatment groups with 8 animals per group were used as outlined below:
   (i). CIA+gavage with oil alone
   (ii). CIA+gavage with oil containing bovine collagen II (10 μg/dose)
   (iii). CIA+gavage with bovine collagen II (20 m/dose)

Clinical scores of arthritis incidence (% of animals in each group affected with arthritis of any severity in any number of joints) and arthritis severity (the severity of disease in each individual mouse) was performed after CIA induction for the duration of the experiment. A standard arthritis scoring system was used as outlined below. Each paw is evaluated for swelling and deformity, scored and a total calculated for each animal.

Score 0: No arthritis
Score 1:1-2 toes affected only
Score 2: 3 or >toes and/or swelling of the paw (metacarpus/metatarsus)
Score 3: swelling of the carpus/tarsus
Score 4: deformity with ankylosis of the carpus/tarsus
3. At the termination of the study (day 21), mice were euthanized, front and rear legs were harvested, fixed in 10% neutral buffered formalin, decalcified in 10% formic acid in 5% formalin and paraffin embedded. Sagittal sections of the right knee joints were stained with Toluidine blue and fast green. Sections were scored by a single blinded observer (CBL) using a standard histopathological grading system (see appendix 1).

As can be seen in FIGS. 1 and 2, a greater reduction in the number of animals suffering from arthritis was observed for the 10 ug dose of collagen in oil, than was achieved for 20 ug of collagen alone. In addition, effects on morphological change

1. A single phase hydrophobic preparation comprising a hydrophilic species, and an amphiphilic component comprising sodium docusate, a phospholipid and a nonionic amphiphile, in an oil phase, wherein the moieties of the hydrophilic species are surrounded by the amphiphilic component with the hydrophilic head groups of the amphiphilic component orientated towards the hydrophilic species and wherein there is no chemical interaction between the amphiphilic component and the hydrophilic species; characterised in that said non-ionic amphiphile has a lipophilic chain comprising 10 to 20 carbons, and a head group comprising 2 to 10 oxyethylene groups or 1 to 3 hydroxyl groups.

2. The preparation of claim 1 wherein said hydrophilic species is selected from peptides, proteins, lipids, sugars, nucleic acids, steroids and/or conjugates of one or more of these agents in combination, and/or a conjugate with at least one medium- or long-chain hydrocarbon tail.

3. A method of manufacture of a hydrophobic preparation containing a hydrophilic species which includes the following steps:
(i) mixing a solution of sodium docusate, a phospholipid and a non-ionic amphiphile dissolved in a hydrophobic solvent with a solution of a hydrophilic species dissolved in an aqueous phase to form an emulsion;
(ii) removing the aqueous phase and hydrophobic solvent; and
(iii) adding an oil phase to the dry residue obtained in (ii).

4. A method as claimed in claim 3 wherein said hydrophobic solvent is selected from cyclohexane, cycloheptane, cyclooctane or mixtures thereof and optionally tertiary butanol.

5. A method as claimed in claim 3 wherein step (ii) is carried out by lyophilisation.

6. The method of claim 3 wherein said hydrophilic species is selected from peptides, proteins, lipids, sugars, nucleic acids, steroids and/or conjugates of one or more of these agents in combination, and/or a conjugate with at least one medium- or long-chain hydrocarbon tail.

7. A method of claim 3 wherein said hydrophilic species is collagen or fragments, derivatives and analogues thereof, which is dissolved in a mixture of acetic acid and dimethyl sulfoxide.

8. A two phase composition comprising an aqueous phase and a single phase hydrophobic preparation of claim 1.

9. A two phase composition of claim 8 wherein said two phase composition is an oil-in water emulsion.

10. A two phase composition of claim 8 wherein said aqueous phase comprises gelatin or albumin.

11. A two phase composition of claim 10 wherein said two phase composition is a microcapsule.

12. A method of forming a two phase composition comprising contacting a preparation of claim 1.

13. The method of claim 12 wherein said aqueous phase comprises gelatin or albumin.

14. A preparation of claim 1 for use in medicine.

15. A composition comprising a preparation of claim 1 and optionally one or more pharmaceutical excipients, diluents or carriers.

16. A vaccine comprising a composition of claim 15.

17. A vaccine of claim 16 adapted for oral administration.

18. A vaccine of claim 17 comprising a capsule.

19. A vaccine of claim 18, wherein said capsule is enterically coated.

20. The vaccine of claim 16 wherein said composition comprises a malaria antigen or an influenza antigen, or an enteric disease pathogen antigen.

21. The pharmaceutical composition of claim 15 adapted for oral, intramuscular or subcutaneous administration.

22. An enterically coated capsule comprising a pharmaceutical composition of claim 15.

23. A composition of claim 15 comprising collagen for use in treating rheumatoid arthritis.

24. A cosmetic formulation comprising preparation of claim 1 and optionally one or more excipients, diluents or carriers.
